(19) United States
(12) Patent Application Publication
(10) Pub. No.: US 2025/0318796 A1
(43) Pub. Date: Oct. 16, 2025

(54) MEDICAL SYSTEM AND RECORDING MEDIUM

(71) Applicant: GE Precision Healthcare LLC, Waukesha, WI (US)

(72) Inventors: Koichi Hoshino, Hino (JP); Yasuhiro Imai, Hino (JP)

(21) Appl. No.: 19/175,685

(22) Filed: Apr. 10, 2025

(30) Foreign Application Priority Data
Apr. 10, 2024 (JP) 2024-063671

(51) Int. Cl. A61B 6/58; A61B 6/00; A61B 6/03

(52) U.S. Cl. CPC A61B 6/582; A61B 6/032; A61B 6/482; A61B 6/5205; A61B 6/5258

(57) ABSTRACT

Methods and systems are described that shorten a time required for calibration. A medical system being a CT system for performing imaging to obtain CT images of a subject, the CT system including one or a plurality of processors, the one or a plurality of processors performing, each time an imaging is performed, selection of calibration data to be used to reconstruct an image from a plurality of calibration data, and determination of an update frequency of each calibration datum based on both a number of times each calibration datum is used and a weighting coefficient according to an imaging purpose of the executed imaging.

### CROSS-REFERENCE TO RELATED APPLICATIONS

This application claims priority to Japanese Application No. 2024-063671, filed on Apr. 10, 2024 the disclosure of which is incorporated herein by reference in its entirety.

### TECHNICAL FIELD

The present invention relates to a medical system for executing beam hardening correction; and a recording medium in which an instruction for controlling the medical system is recorded.

### BACKGROUND

A CT system is known as a medical device that noninvasively images a subject. CT systems can acquire tomographic images of a subject in a short scanning time and therefore are widely used in hospitals and other medical facilities.

The CT system applies a prescribed voltage to a cathode-anode tube of an X-ray tube to generate X-rays. The generated X-rays penetrate the subject and are detected by a detector. The CT system reconstructs a CT image of the subject based on data detected by the detector.

### SUMMARY

Single energy CT (SECT) is a well-known imaging technique for CT systems. SECT is a method for obtaining a CT image of a subject by applying a prescribed voltage (e.g., 120 kV) to a cathode-anode tube of an X-ray tube to generate X-rays. However, in SECT, CT values may be close even for different substances, and identification of different substances may be difficult.

Therefore, DECT (Dual Energy CT) technology is being researched and developed. DECT is a technology that can use X-rays in different energy regions to distinguish between substances, and can acquire images that are useful for diagnosis in clinical settings, and thus is beginning to come into widespread use. In the DECT technology, a kV switching technology is known that switches the tube voltage of the X-ray tube between a low tube voltage and a high tube voltage.

Furthermore, parts of a CT system deteriorate over time as the system is used for a long period of time, and as a result, the calibration data used in CT system calibration deviates from the ideal value. Therefore, in the CT system, a calibration scan is periodically executed to acquire calibration data necessary for calibration of the CT system.

For example, the material decomposition accuracy of kV switching by DECT depends on the rotation speed of the gantry, the cone angle, the tube current, and the like. For this reason, in calibration, a plurality of combinations of values of these parameters (rotation speed, cone angle, and tube current) are prepared in advance, a calibration scan is executed for each preset, and calibration data is found. Therefore, it is necessary to execute a plurality of calibration scans in one calibration, and there is a problem that the time required for the calibration becomes long.

Technology that can reduce the time required for one calibration is desired.

A first aspect of the present invention is a medical system for performing an imaging to obtain medical images of a subject, the medical system including one or more processors, wherein the one or more processors, each time an imaging is performed, select calibration data to be used to reconstruct an image from a plurality of calibration data, and determine an update frequency for each calibration data based on the number of times each calibration data is used and a weighting coefficient according to the imaging purpose of the performed imaging.

A second aspect of the present invention is a non-transitory computer-readable storage medium included in a medical system or communicable with the medical system, wherein the non-transitory computer-readable storage medium cause the one or more processors to execute the following operations when instructions stored in the storage medium are executed by the one or more processors: selecting calibration data to be used for reconstructing an image from among a plurality of calibration data each time imaging is performed; and determining an update frequency for each calibration data based on the number of times each calibration data is used and a weighting coefficient according to the imaging purpose of the performed imaging.

In the present invention, the update frequency of each piece of calibration data is determined based on the number of times of use of each piece of calibration data and the weighting coefficient according to the imaging purpose of the executed imaging. Therefore, in the case of calibration data that is used a small number of times or calibration data is used in imaging with a small weighting coefficient, the update frequency of the calibration data can be lowered, and thus the time required for one calibration can be shortened.

### BRIEF DESCRIPTION OF THE DRAWINGS

FIG. 1 is a block diagram of a CT system 10 of the present embodiment.

FIG. 2 is an explanatory diagram of calibration.

FIG. 3 is an explanatory diagram of steps executed on a medical examination day 1.

FIG. 4 is a diagram illustrating a state in which an imaging 1 is executed.

FIG. 5 is flowchart of a method for determining the update frequency of calibration data D1, D2 and D3.

FIG. 6 is a diagram illustrating a table for describing each step ST1 to ST4 in FIG. 5.

FIG. 7 is a diagram illustrating the index values and update frequencies of the calibration data D1, D2, and D3 stored in association with the imaging 1.

FIG. 8 is a diagram illustrating a state in which an imaging 2 is executed.

FIG. 9 is a table for describing each step of FIG. 5 when the imaging 2 is executed.

FIG. 10 is a diagram illustrating the index values and update frequencies of the calibration data D1, D2, and D3 stored in association with the imaging 2.

FIG. 11 is a diagram illustrating a state in which an imaging 3 is executed.

FIG. 12 is a diagram illustrating a table for describing each step of FIG. 5 when the imaging 3 is executed.

FIG. 13 is a diagram illustrating the index values and update frequencies of the calibration data D1, D2, and D3 stored in association with the imaging 3.

FIG. 14 is a diagram illustrating a state in which an imaging 4 is executed.

FIG. 15 is a diagram illustrating a table for describing each step of FIG. 5 when the imaging 4 is executed.

FIG. 16 is a diagram illustrating the index values and update frequencies of the calibration data D1, D2, and D3 stored in association with the imaging 4.

FIG. 17 is a diagram illustrating a state in which an imaging 5 is executed.

FIG. 18 is a diagram illustrating a table for describing each step of FIG. 5 when the imaging 5 is executed.

FIG. 19 is a diagram illustrating the index values and update frequencies of the calibration data D1, D2, and D3 stored in association with the imaging 5.

FIG. 20 is an explanatory diagram of medical examination day 2.

FIG. 21 is an explanatory diagram of step ST22.

FIG. 22 is a diagram illustrating a table for describing each step of FIG. 5 when the imaging 6 is executed.

FIG. 23 is a diagram illustrating the index values and update frequencies of the calibration data D1, D2, and D3 stored in association with the imaging 6.

FIG. 24 is a diagram illustrating the index values and update frequencies obtained in a final imaging p on medical examination day 2.

FIG. 25 is an explanatory diagram of medical examination day 3.

### DETAILED DESCRIPTION OF THE DRAWINGS

An embodiment for carrying out the invention will be described below, but the present invention is not limited to the following embodiment.

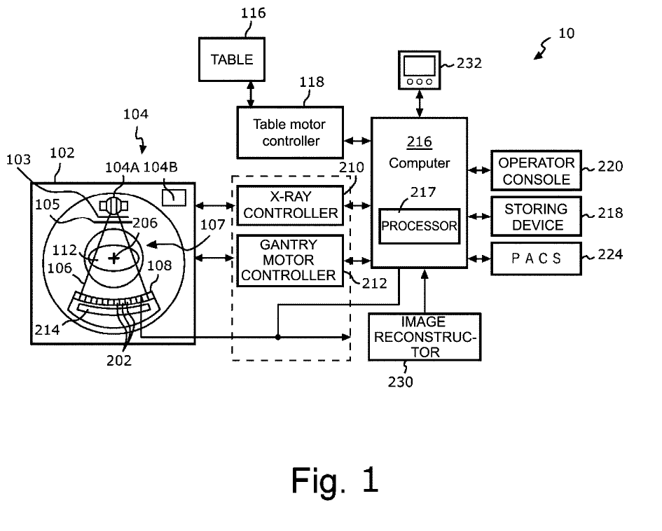

FIG. 1 is a block diagram of a CT system 10 of the present embodiment. The CT system 10 includes a gantry 102 and a table 116. The gantry 102 includes a bore 107, and a subject 112 is transported through the bore 107, and then the subject 112 is scanned. The gantry 102 is equipped with an X-ray generation device 104, a filter part 103, a pre-collimator 105, a detector 108, and the like.

The X-ray generation device 104 includes an X-ray tube 104A and a generator 104B. The generator 104B supplies power to the X-ray tube 104A. The X-ray tube 104A outputs X-rays when a prescribed voltage is applied to a cathode-anode tube. The X-ray tube 104 is configured to be rotatable on a path centered on a rotation axis 206 within the XY plane. Herein, the Z direction represents the body-axis direction, the Y direction represents the vertical direction (the height direction of the table 116), and the X direction represents the direction perpendicular to the Z and Y directions. In the present embodiment, the X-ray tube 104A supports a kV switching scheme in which the tube voltage applied to the X-ray tube can be alternatingly switched between a first tube voltage and a second tube voltage. Note that in the present embodiment, the CT system 10 includes one X-ray tube 104A, but may include two X-ray tubes 104A.

The filter part 103 includes, for example, a flat plate filter and/or a bow-tie filter. The pre-collimator 105 is a member that narrows the X-ray irradiation range such that X-rays are not irradiated in unwanted regions. The detector 108 includes a plurality of detector elements 202. The plurality of detector elements 202 detect an X-ray beam 106 that is irradiated from the X-ray tube 104A and passes through the subject 112, such as a patient or the like. Therefore, the X-ray detector 108 can acquire projection data for each view.

The projection data detected by the detector 108 is collected by a DAS 214. The DAS 214 executes prescribed processing, including sampling, digital conversion, and the like, on the collected projection data. The processed projection data is transmitted to a computer 216. The computer 216 stores the data from the DAS 214 in a storing device 218. The storage device 218 includes one or more storage media that record programs, instructions, and the like to be executed by the processor. The storage medium may be, for example, one or more non-transitory computer-readable storage media. The storing device 218 may include, for example, hard disk drives, floppy disk drives, compact disc read/write (CD-R/W) drives, digital versatile disk (DVD) drives, flash drives, and/or solid state recording drives.

The computer 216 includes one or a plurality of processors 217. The computer 216 uses one or a plurality of processors to output commands and parameters to the DAS 214, X-ray controller 210, and/or gantry motor controller 212, to control system operations such as data acquisition and/or processing. Furthermore, the computer 216 uses one or a plurality of processors to execute various processes such as signal processing, data processing, image processing, and the like in each step of the flow described later. Note that in FIG. 1, one or a plurality of the processors 217 are included in the computer 216, but one or a plurality of the processors 217 may be provided so as to be distributed between the computer 216 and another constituent element (for example, X-ray controller 210, gantry motor controller 212, table controller 118, or the like).

An operator console 220 is linked to the computer 216. An operator can enter prescribed operator inputs related to the operation of the CT system 10 into the computer 216 by operating the operator console 220. The computer 216 receives an operator input, including a command and/or scan parameter, via the operator console 220 and controls system operation based on the operator input. The operator console 220 can include a keyboard (not depicted) or touch screen for the operator to specify a command and/or scan parameter.

The X-ray controller 210 controls the X-ray generation device 104 based on an instruction from the computer 216. Furthermore, the gantry motor controller 212 also controls a gantry motor to rotate a constituent element, such as the X-ray tube 104A, detector 108, and the like, based on instruction from the computer 216.

FIG. 1 depicts only one operator console 220, but two or more operator consoles may be linked to the computer 216. Furthermore, the CT system 10 may also allow a plurality of remotely located displays, printers, workstations, and/or similar devices to be linked via, for example, a wired and/or wireless network.

In one embodiment, for example, the CT system 10 may include a Picture Archiving and Communication System (PACS) 224, or may be linked to the PACS 224. In a typical implementation, a PACS 224 may be linked to a remote system such as a radiology department information system, hospital information system, and/or internal or external network (not depicted) or the like.

The computer 216 provides an instruction to a table motor controller 118 to control the table 116. The table motor controller 118 can control the table motor so as to move the table 116 based on the instructions received. For example, the table motor controller 118 can move the table 116 such that the subject 112 is positioned appropriately for imaging.

As mentioned above, the DAS 214 samples and digitally converts the projection data acquired by the detector elements 202. The image reconstructor 230 then reconstructs the CT image using the sampled and digitally converted data. The image reconstructor 230 includes one or a plurality of processors, which can execute image reconstruction processing. In FIG. 1, the image reconstructor 230 is depicted as a separate component from the computer 216, but the image reconstructor 230 may form a part of the computer 216. Furthermore, the computer 216 may also perform one or a plurality of functions of the image reconstructor 230. Furthermore, the image reconstructor 230 may be positioned away from the CT system 10 and operatively connected to the CT system 10 using a wired or wireless network.

The image reconstructor 230 can store the reconstructed image in the storing device 218. The image reconstructor 230 may also transmit the reconstructed image to the computer 216. The computer 216 can transmit the reconstructed image and/or patient information to a display device 232 communicatively linked to the computer 216 and/or image reconstructor 230.

Non-transitory computer-readable storage media included in or in communication with the CT system 10 may store instructions for executing the various methods, steps, and processes described herein. The instructions may be stored on a single storage medium or distributed across multiple storage media. Also, the instructions may be stored on an external storage device accessible by the CT system. One or more processors provided in the CT system 10 execute the various methods, steps, and processes described in the present specifications in accordance with the instructions recorded on a recording medium.

The CT system 10 is configured as described above. Parts of a CT system deteriorate over time as the system is used for a long period of time, and as a result, properties of parts used in the CT system fluctuate. Therefore, in the CT system, a calibration scan is periodically executed to acquire calibration data for calibrating fluctuation of properties of the CT system.

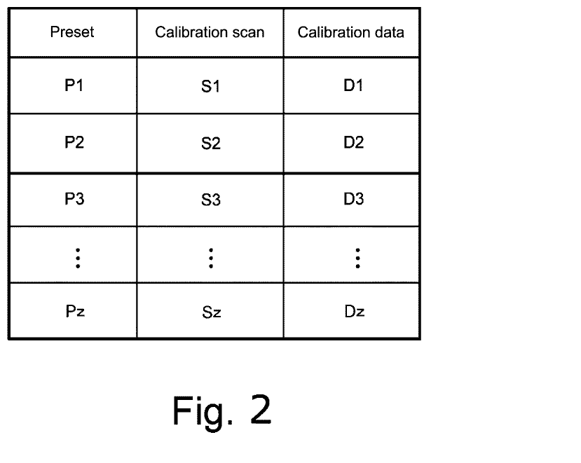

FIG. 2 is an explanatory diagram of calibration. In the calibration of the kV switching method, for example, z combinations P1 to Pz are prepared as combinations of values of three parameters (rotation speed, cone angle, tube current). Then, z combinations P1 to Pz are preset and stored in the CT system, and a calibration scan is executed for each combination. By executing the calibration scan, X-rays are detected by the detector 108. The processor generates calibration data based on data of X-rays detected by the detector 108. FIG. 2 illustrates an example in which z calibration datasets D1 to Dz are generated for z presets P1 to Pz.

However, as the number of presets increases, the number of calibration scans that must be executed also increases, resulting in a problem in which a great deal of time is required for calibration. For example, when three rotation speeds (r1, r2, and r3) are considered as the rotation speed, three cone angles (c1, c2, and c3) are considered as the cone angle, and three tube currents (a1, a2, and a3) are considered as the tube current, 27 combinations S1 to S27 are prepared as presets, and the calibration scan is executed for each of the combinations S1 to S27. Therefore, it is necessary to execute a 27 calibration scans in one calibration, that is, to acquire 27 items of calibration data, and there is a problem that the time required for the calibration becomes long. Therefore, the CT system of the present embodiment is configured to be able to shorten the time required for one calibration. The present embodiment will be described below.

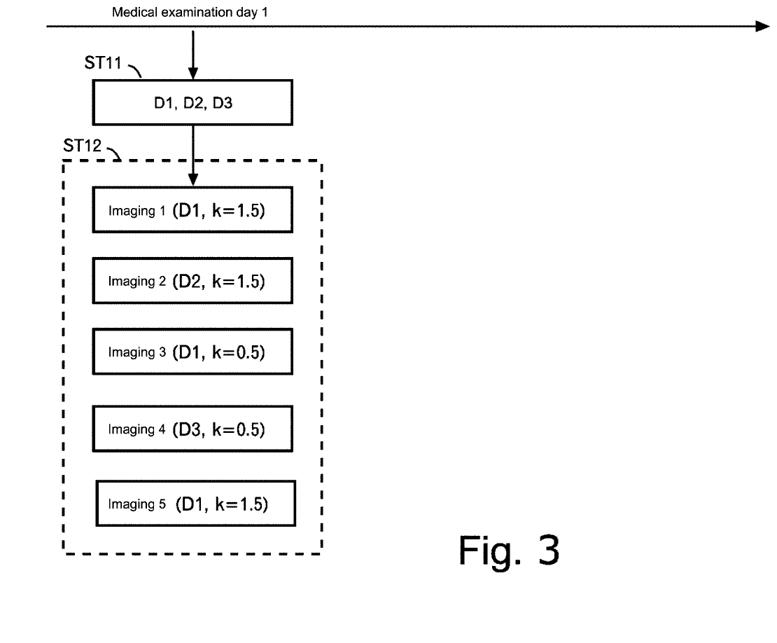

FIG. 3 is an explanatory diagram of steps executed on a medical examination day 1. In step ST11, calibration is executed. In the calibration, z pieces of calibration data are generated as described with reference to FIG. 2. However, in the following description, in order to facilitate understanding of the present embodiment, only three calibration data D1, D2, and D3 will be considered as generated calibration data. The processor stores the generated calibration data D1, D2, and D3 in the storage device. After the calibration data D1, D2, and D3 are stored, the process proceeds to step ST12.

In step ST12, a subject body scan is performed. In the box of step ST12, examples of imaging actually executed in the examination are shown in chronological order. In FIG. 3, for convenience of description, an example in which the imaging 1 to imaging 5 are executed is illustrated. In the imaging 1 to imaging 5, calibration data used at the time of imaging and a weighting coefficient k according to the purpose of imaging are shown. For example, “D1” is listed in the box of the imaging 1. This means that in the imaging 1, the processor has selected the calibration data D1 from among the calibration data D1 to D3 as the calibration data to be used for image reconstruction. In addition, “k=1.5” is listed in the box of the imaging 1. Here, k represents a weighting coefficient according to the purpose of imaging. The weighting coefficient k is a value set for each imaging protocol and reflects the purpose of imaging. For example, there is a case where high image quality is required depending on a imaging purpose, and in this case, the weighting coefficient k is set to a high value. On the other hand, there is a case where the required image quality is not so high depending on the purpose of imaging, and in this case, the weighting coefficient k is set to a low value. In the present embodiment, for convenience of explanation, two values, that is, “1.5” and “0.5,” are considered as the weighting coefficient k, and the weighting coefficient k is set to 1.5 or 0.5 according to the purpose of imaging. In the imaging 1, the weighting coefficient k is k=1.5.

Referring to the box of the imaging 2, “D2” is listed in the box of the imaging 2. This means that in the imaging 2, the processor has selected the calibration data D1 from among the calibration data D1 to D3 as the calibration data to be used for image reconstruction. In addition, “k=1.5” is listed in the box of the imaging 2. Therefore, in the imaging 2, the weighting coefficient k is k=1.5.

Referring to the box of the imaging 3, “D1” is listed in the box of the imaging 3. Therefore, the calibration data D1 is selected as the calibration data used for the image reconstruction. In addition, “k=0.5” is listed in the box of the imaging 3. Therefore, in the imaging 3, the weighting coefficient k is k=0.5.

Referring to the box of imaging 4, “D3” is listed in the box of imaging 4. This means that in the imaging 4, the processor has selected the calibration data D3 from among the calibration data D1 to D3 as the calibration data to be used for image reconstruction. In addition, “k=0.5” is listed in the box of the imaging 4. Therefore, in the imaging 4, the weighting coefficient k is k=0.5.

Referring to the box of the imaging 5, “D1” is listed in the box of the imaging 6. Therefore, the calibration data D1 is selected as the calibration data used for the image reconstruction. In addition, “k=1.5” is listed in the box of the imaging 5. Therefore, in the imaging 5, the weighting coefficient k is k=1.5.

The processor determines an update frequency of each piece of calibration data in consideration of a use situation of the calibration data used in the imaging every time each imaging 1 to 5 is executed. The update frequency of the calibration data represents the degree to which the update of the calibration data is repeatedly executed. For example, in the present embodiment, the following three frequencies are considered as the update frequency of the calibration data: (1) Update calibration data every day, (2) Update calibration data every other day, and (3) Update calibration data every three days. Therefore, the processor determines the update frequency (whether to update every day, every other day, or every three days) for each of the calibration data D1, D2, and D3. A method of determining the update frequency of the calibration data will be specifically described below.

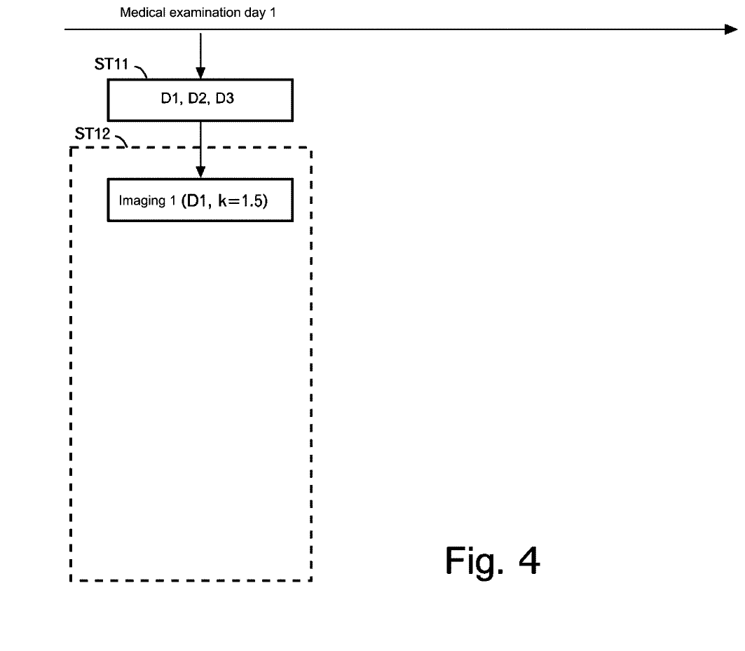

The operator operates the console to input a signal for selecting a protocol corresponding to the imaging purpose of the imaging 1. When this signal is input, the processor selects from among a plurality of protocols a protocol corresponding to the imaging purpose of the imaging 1. A weighting coefficient corresponding to the imaging purpose is set for each protocol, and the processor can read the value of the weighting coefficient as necessary. After the protocol is selected, the imaging 1 is executed as illustrated in FIG. 4. When the imaging 1 is executed, the processor determines update frequencies of the calibration data D1, D2, and D3. The update frequency determination method will be described below.

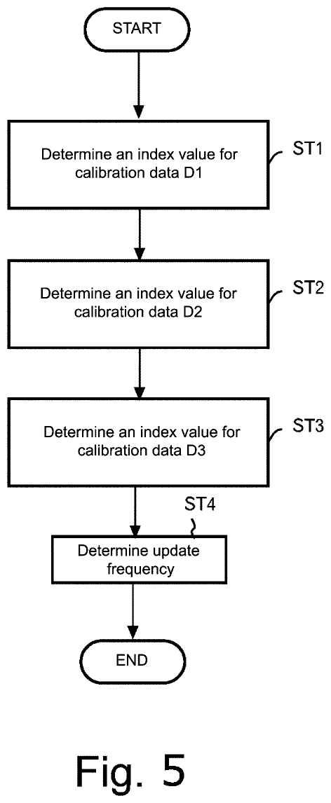

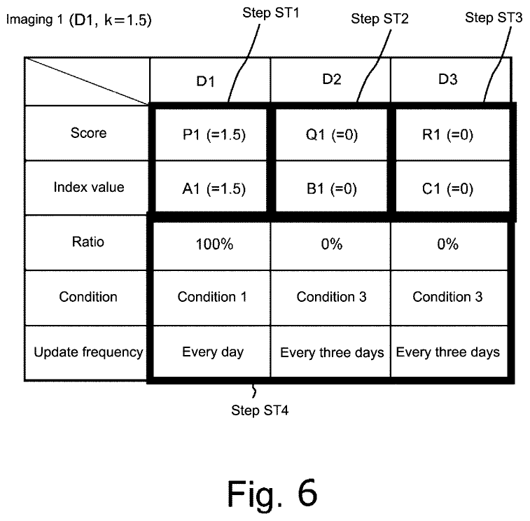

FIG. 5 is a flowchart of a method for determining the update frequencies of the calibration data D1, D2, and D3 when the imaging 1 is executed, and FIG. 6 is a diagram illustrating a table for explaining steps ST1 to ST4 of FIG. 5. Each step is described below.

In step ST1, an index value serving as a criterion for determining the update frequency of the calibration data D1 is determined. Specifically, the index value is determined as follows. In step ST1, first, a score P1 of the calibration data D1 for the imaging 1 is determined. When the calibration data D1 is not used in the imaging 1, the processor assigns P1=0 to the score P1. On the other hand, when the calibration data D1 is used in the imaging 1, the processor assigns the value of the weighting coefficient k set for the selected protocol to the score P1. The calibration data D1 is used in the imaging 1, the processor assigns the value of the weighting coefficient k to the score P1. In the imaging 1, the weighting coefficient k of the imaging purpose is k (=1.5). Therefore, the processor assigns P1=1.5 to the score P1.

After determining the score P1, the processor determines an index value A1 that serves as a criterion for determining the update frequency of the calibration data D1, based on the score P1. In the imaging 1, the value of the score P1 is adopted as the initial value of the index value A1. Therefore, A1=1.5 is determined. Once the index value A1 has been calculated, the process proceeds to step ST2.

In step ST2, an index value is determined as a criterion for determining the update frequency of the calibration data D2. Specifically, the index value is determined as follows. In step ST2, first, a score Q1 of the calibration data D2 for the imaging 1 is determined. When the calibration data D2 is not used in the imaging 1, the processor assigns Q1=0 to the score Q1. On the other hand, when the calibration data D2 is used in the imaging 1, the processor assigns the value of the weighting coefficient k to the score Q1. Since the calibration data D2 is not used in the imaging 1, the processor assigns Q1=0 to the score Q1.

After determining the score Q1, the processor determines an index value B1 serving as a criterion for determining the update frequency of the calibration data D2 based on the score Q1. In the imaging 1, the value of the score Q1 is adopted as the initial value of the index value B1. Therefore, B1=0 is determined. Once the index value B1 has been calculated, the process proceeds to step ST3.

In step ST3, an index value serving as a criterion for determining the update frequency of the calibration data D3 is determined. Specifically, the index value is determined as follows. In step ST3, first, a score R1 of the calibration data D3 for the imaging 1 is determined. When the calibration data D3 is not used in the imaging 1, the processor assigns R1=0 to the score R1. On the other hand, when the calibration data D3 is used in the imaging 1, the processor assigns the value of the weighting coefficient k to the score R1. Since the calibration data D3 is not used in the imaging 1, the processor assigns R1=0 to the score R1.

After determining the score R1, the processor determines an index value C1 serving as a criterion for determining the update frequency of the calibration data D3 based on the score R1. In the imaging 1, the value of the score R1 is adopted as the initial value of the index value C1. Therefore, C1=0 is determined.

Therefore, in the imaging 1, the index value A1=1.5, the index value B1=0, and the index value C1=0 are determined. After calculating index values A1, B1, and C1, processing proceeds to step ST4.

In step ST4, the processor determines update frequencies of the calibration data D1, D2, and D3 based on the index values A1, B1, and C1. Specifically, the update frequencies of the calibration data D1, D2, and D3 are determined as follows. First, the processor calculates a ratio L (%) of each index value to the total value of the index values A1, B1, and C1. Here, since the index value A1=1.5, the index value B1=0, and the index value C1=0, the total value of the index values is 1.5. Therefore, the ratio L of each index value is 100% for the index value A1, and 0% for the index values B1 and C1. Then, the processor compares the ratio of each index value to a threshold value. In the present embodiment, two thresholds, that is, a threshold TH1 and a threshold TH2 smaller than the threshold TH1 are considered. In the following description, the thresholds TH1 and TH2 are set to 60% and 20%, respectively, for convenience of description. Then, the processor determines which one of the following conditions 1 to 3 is satisfied by the ratio P of each index value with respect to the threshold TH1 and the threshold TH2.
(Condition 1) P≥TH1, that is, P≥60%
(Condition 2) TH1>P≥TH2, that is, 60%>P≥20%
(Condition 3) TH2>P, that is, 20%>P
   When condition 1 is satisfied, the processor determines to update the calibration data every day, when condition 2 is satisfied, the processor determines to update the calibration data every other day, and when condition 3 is satisfied, the processor determines to update the calibration data every three days.

Here, the ratio L of the index value A1 is L=100%. Therefore, since the ratio L of the index value A1 corresponds to the condition 1, the processor determines that the calibration data D1 is updated every day.

On the other hand, the ratio L of the index values B1 and C1 is L=0%. Therefore, since the ratio L of the index values B1 and C1 corresponds to the condition 3, the processor determines that the calibration data D2 and D3 are updated every three days. After the update frequency is determined, the flow of FIG. 5 ends.

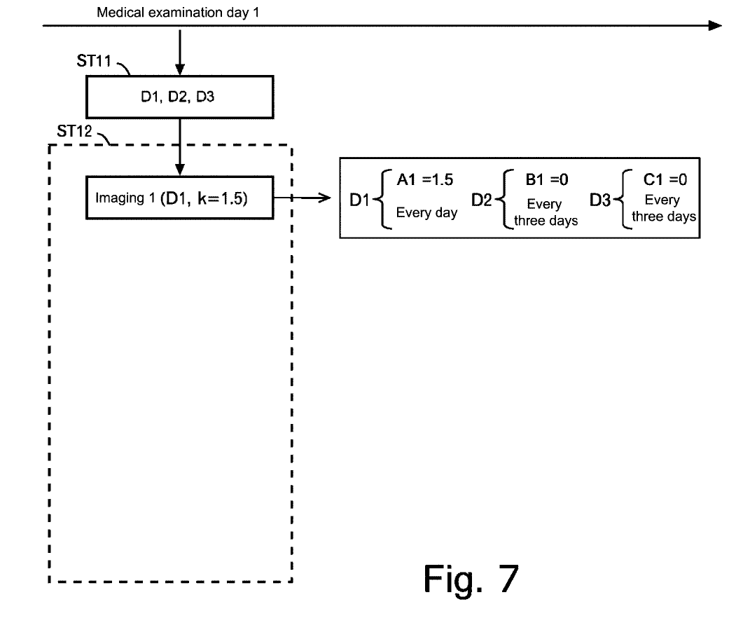

After determining the update frequency, the processor stores the index value and the update frequency obtained by the flow of FIG. 5 in the storage device. FIG. 7 illustrates the index values and update frequencies of the calibration data D1, D2, and D3 stored in association with the imaging 1.

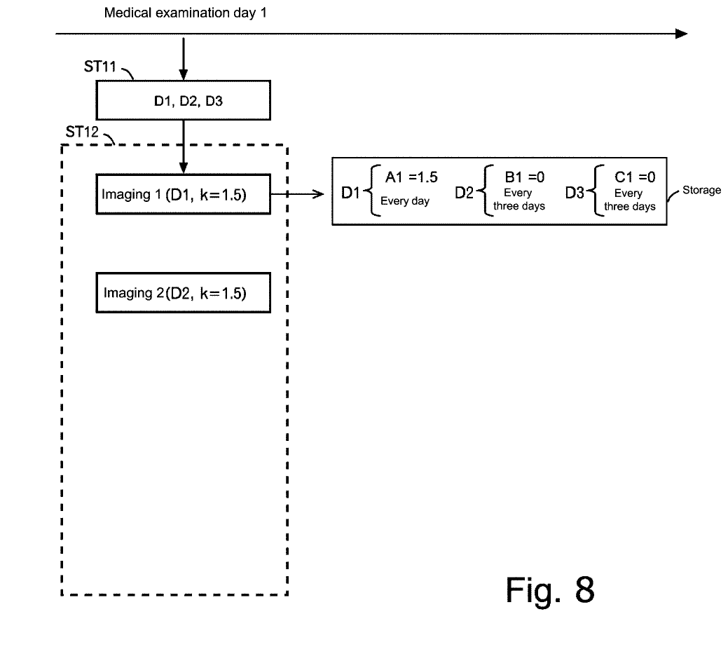

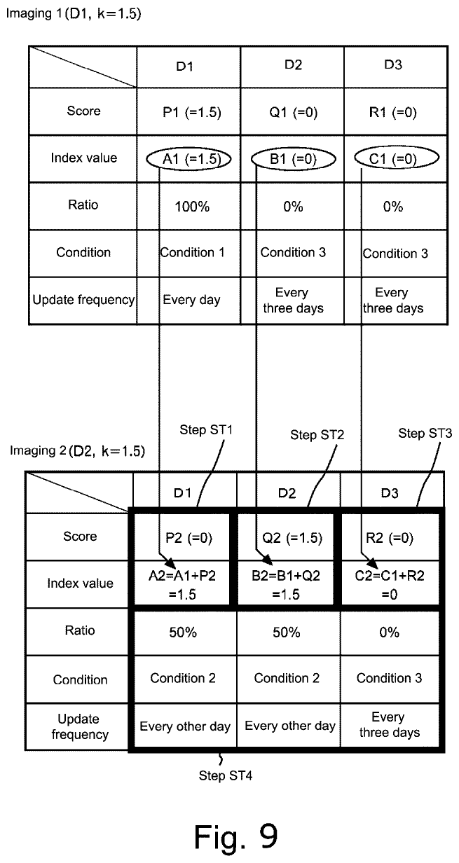

Next, the operator operates the console to input a signal for selecting a protocol corresponding to the imaging purpose of the imaging 2. When this signal is input, the processor selects a protocol corresponding to the imaging purpose of the imaging 2 from among a plurality of protocols. After the program is selected, imaging 2 is executed as illustrated in FIG. 8. When the imaging 2 is executed, the processor determines the update frequencies of the calibration data D1, D2, and D3. A method of determining the update frequency will be described below with reference to FIG. 9 together with the flow of FIG. 5.

FIG. 9 is a table for describing each step of FIG. 5 when the imaging 2 is executed. In FIG. 9, not only is the table of the imaging 2 shown but also the table of the imaging 1.

In step ST1, an index value serving as a criterion for determining the update frequency of the calibration data D1 is determined. Specifically, the index value is determined as follows. In step ST1, first, the score P2 of the calibration data D1 for the imaging 2 is determined. When the calibration data D1 is not used in the imaging 2, the processor assigns P2=0 to the score P2. On the other hand, when the calibration data D1 is used in the imaging 2, the processor assigns the value of the weighting coefficient k to the score P2. Since the calibration data D1 is not used in the imaging 2, the processor assigns P2=0 to the score P2.

After determining the score P2, the processor determines an index value A2 serving as a criterion for determining the update frequency of the calibration data D1 based on the score P2. Specifically, the processor calculates an index value A2 (=A1+P2) by adding the score P2 to the index value A1 determined in the imaging 1. A2 is a value expressed by the following formula.

$$A 2 + A 1 + P 2 = 1.5 + 0 = 1.5$$

Therefore, the index value A2 is calculated as A2=1.5. Once the index value A2 has been calculated, the process proceeds to step ST2.

In step ST2, an index value is determined as a criterion for determining the update frequency of the calibration data D2. Specifically, the index value is determined as follows. In step ST2, first, the score Q2 of the calibration data D2 for the imaging 2 is determined. When the calibration data D2 is not used in the imaging 2, the processor assigns Q2=0 to the score Q2. On the other hand, when the calibration data D2 is used in the imaging 2, the processor assigns the value of the weighting coefficient k to the score Q2. Since the calibration data D2 is used in the imaging 2, the processor assigns the value of the weighting coefficient k to the score Q2. In the imaging 2, the weighting coefficient k of the imaging purpose is k (=1.5). Therefore, the processor assigns Q2=1.5 to the score Q2.

When the score Q2 is determined, the processor determines the index value B2 serving as a criterion for determining the update frequency of the calibration data D2 based on the score Q2. Specifically, the processor calculates an index value B2 (=B1+Q2) by adding the score Q2 to the index value B1 determined in the imaging 1. B2 is a value expressed by the following formula.

$$B 2 = B 1 + Q 2 = 1.5 + 0 = 1.5$$

Therefore, the index value B2 is calculated as B2=1.5. Once the index value B2 has been calculated, the process proceeds to step ST3.

In step ST3, the processor determines an index value serving as a criterion for determining the update frequency of the calibration data D3. Specifically, the index value is determined as follows. In step ST3, the processor determines a score R2. When the calibration data D3 is not used in the imaging 2, the processor assigns R2=0 to the score R2. On the other hand, when the calibration data D3 is used in the imaging 2, the processor assigns the value of the weighting coefficient to the score R2. Since the calibration data D3 is not used in the imaging 2, the processor determines the score R2 as R2=0.

When the score R2 is determined, the processor determines the index value C2 serving as a criterion for determining the update frequency of the calibration data D3 based on the score R2. Specifically, the processor calculates an index value C2 (=C1+R2) by adding the score R2 to the index value C1 determined in the imaging 1. C2 is a value expressed by the following formula.

$$C 2 = C 1 + R 2 = 0 + 0 = 0$$

Therefore, the index value C2 is calculated as C2=0.

Therefore, at the time point when the imaging 2 ends, the index value A2=1.5, the index value B2=1.5, and the index value C2=0 are determined as the latest index values. After calculating index values A2, B2, and C2, processing proceeds to step ST4.

In step ST4, the processor determines update frequencies of the calibration data D1, D2, and D3 based on the index values A2, B2, and C2. Specifically, the update frequencies of the calibration data D1, D2, and D3 are determined as follows. First, the processor calculates a ratio L (%) of each index value to the total value of the index values A2, B2, and C2. Here, since the index value A2=1.5, the index value B2=1.5, and the index value C2=0, the total value of the index values is 3.0. Therefore, the ratio L of each index value is 50% for the index value A2, 50% for the index value B2, and 0% for the index value C2. Then, the processor compares the ratios of the respective index values with the thresholds, and determines which condition among the conditions 1 to 3 the ratios P of the respective index values satisfy with respect to the threshold TH1 and the threshold TH2.

Here, the ratio L of the index value A2 is L=50%. Therefore, since the ratio L of the index value A2 corresponds to the condition 2, the processor determines that the calibration data D1 is updated every other day. The ratio L of the index value B2 is L=50%. Therefore, since the ratio L of the index value B2 corresponds to the condition 2, the processor determines that the calibration data D2 is updated every other day. Further, the ratio L of the index value C2 is L=0%. Therefore, since the ratio L of the index value B2 corresponds to the condition 3, the processor determines that the calibration data D2 is updated every three days. After the update frequency is determined, the flow of FIG. 5 ends.

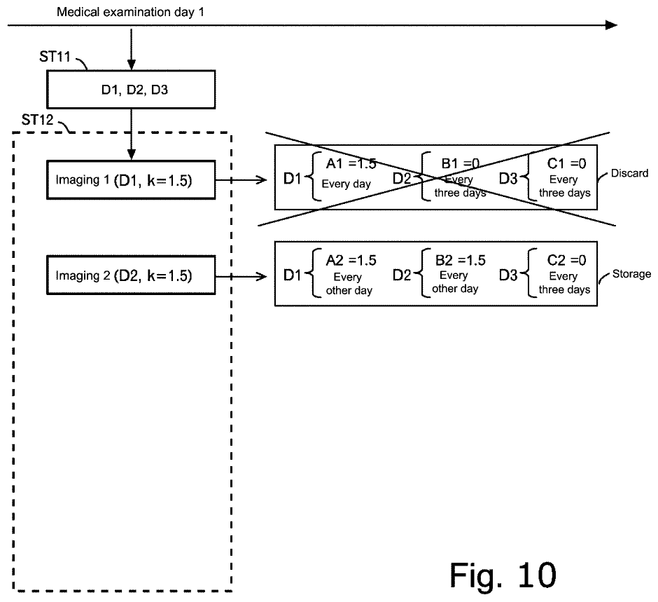

After determining the update frequency, the processor stores the index value and the update frequency obtained by the flow of FIG. 5 in the storage device. FIG. 10 illustrates the index values and update frequencies of the calibration data D1, D2, and D3 stored in association with the imaging 2. Note that the index value and the update frequency obtained in the previous imaging 1 are discarded because they are old information.

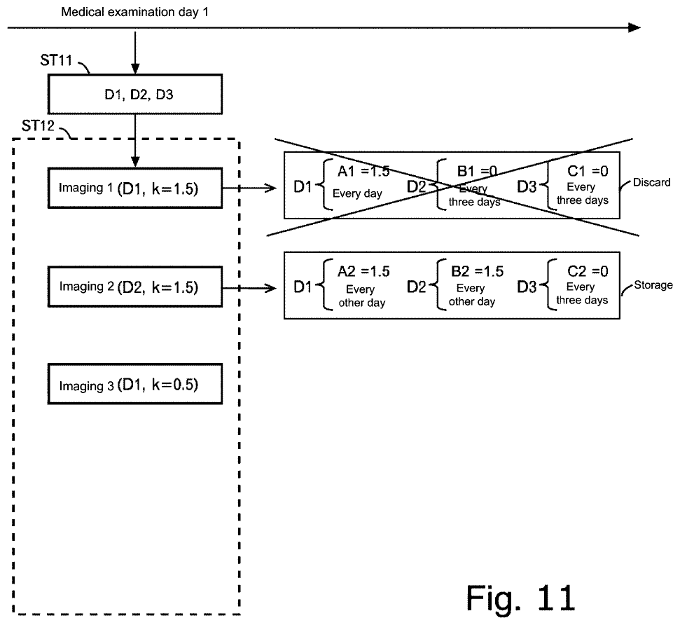

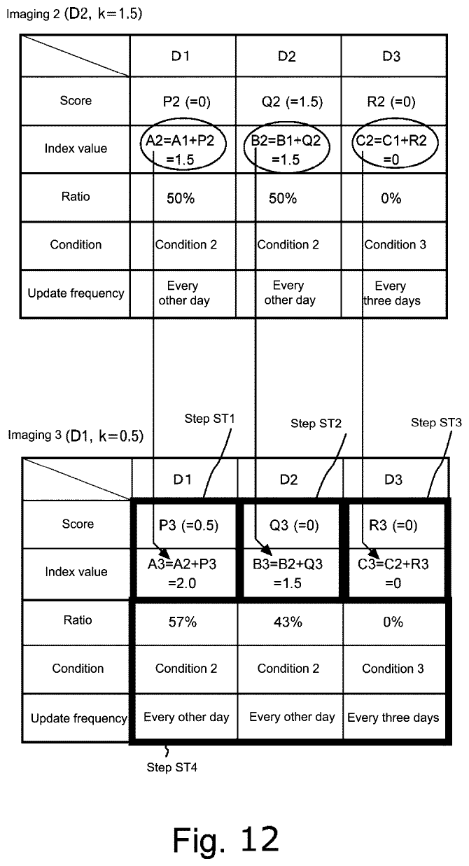

Next, the operator operates the console to input a signal for selecting a protocol corresponding to the imaging purpose of the imaging 3. When this signal is input, the processor selects a protocol corresponding to the imaging purpose of the imaging 3 from among a plurality of protocols. After the program is selected, the imaging 3 is executed as illustrated in FIG. 11. When the imaging 3 is executed, the processor determines the update frequencies of the calibration data D1, D2, and D3. A method of determining the update frequency will be described below with reference to FIG. 12 together with the flow of FIG. 5.

FIG. 12 is a diagram illustrating a table for describing each step of FIG. 5 when imaging 3 is executed. In FIG. 12, not only is the table of the imaging 3 shown but also the table of the imaging 2.

In step ST1, an index value serving as a criterion for determining the update frequency of the calibration data D1 is determined. Specifically, the index value is determined as follows. In step ST1, first, a score P3 of the calibration data D1 for the imaging 3 is determined. When the calibration data D1 is not used in the imaging 3, the processor assigns P3=0 to the score P3. On the other hand, when the calibration data D1 is used in the imaging 3, the processor assigns the value of the weighting coefficient k to the score P3. Since the calibration data D1 is used in the imaging 3, the processor assigns the value of the weighting coefficient k to the score P3. In the imaging 3, the weighting coefficient k for the imaging purpose is k (=0.5). Therefore, the processor assigns P3=0.5 to the score P3.

After determining the score P3, the processor determines an index value A3 serving as a criterion for determining the update frequency of the calibration data D1 based on the score P3. Specifically, the processor calculates an index value A3 (=A2+P3) by adding the score P3 to the index value A2 determined in the imaging 2. A3 is a value expressed by the following formula.

$$A 3 = A 2 + P 3 = 1.5 + 0.5 = 2.$$

Therefore, the index value A3 is calculated as A3=2.0. After the index value A3 has been calculated, the process proceeds to step ST2.

In step ST2, an index value is determined as a criterion for determining the update frequency of the calibration data D2. Specifically, the index value is determined as follows. In step ST2, first, a score Q3 of the calibration data D2 for the imaging 3 is determined. When the calibration data D2 is not used in the imaging 3, the processor assigns Q3=0 to the score Q3. On the other hand, when the calibration data D2 is used in the imaging 3, the processor assigns the value of the weighting coefficient k to the score Q3. In the imaging 3, since the calibration data D2 is not used, the processor assigns Q3=0 to the score Q3.

When the score Q3 is determined, the processor determines the index value B3 serving as a criterion for determining the update frequency of the calibration data D3 based on the score Q3. Specifically, the processor calculates an index value B3 (=B2+Q3) by adding the score Q3 to the index value B2 determined in the imaging 2. B3 is a value expressed by the following formula.

$$B 3 = B 2 + Q 3 = 1.5 + 0 = 1.5$$

Therefore, the index value B3 is calculated as B3=1.5. Once the index value B3 has been calculated, the process proceeds to step ST3.

In step ST3, the processor determines an index value serving as a criterion for determining the update frequency of the calibration data D3. Specifically, the index value is determined as follows. In step ST3, the processor determines a score R3. When the calibration data D3 is not used in the imaging 3, the processor assigns R3=0 to the score R3. On the other hand, when the calibration data D3 is used in the imaging 3, the processor assigns the value of the weighting coefficient to the score R3. Since the calibration data D3 is not used in the imaging 3, the processor determines the score R3 as R3=0.

When the score R3 is determined, the processor determines the index value C3 serving as a criterion for determining the update frequency of the calibration data D3 based on the score R3. Specifically, the processor calculates an index value C3 (=C2+R3) by adding the score R3 to the index value C2 determined in the imaging 2. C3 is a value expressed by the following formula.

$$C 3 = C 2 + R 3 = 0 + 0 = 0$$

Therefore, the index value C3 is determined as C3=0.

Therefore, at the time point when the imaging 3 ends, the index value A3=2.0, the index value B3=1.5, and the index value C3=0 are determined as the latest index values. After calculating index values A3, B3, and C3, processing proceeds to step ST4.

In step ST4, the processor determines update frequencies of the calibration data D1, D2, and D3 based on the index values A3, B3, and C3. Specifically, the update frequencies of the calibration data D1, D2, and D3 are determined as follows. First, the processor calculates a ratio L (%) of each index value to the total value of the index values A3, B3, and C3. Here, since the index value A3=2.0, the index value B3=1.5, and the index value C2=0, the total value of the index values is 3.5. Therefore, the ratio L of each index value is 57% for the index value A3, 43% for the index value B3, and 0% for the index value C3. Then, the processor compares the ratios of the respective index values with the thresholds, and determines which condition among the conditions 1 to 3 the ratios P of the respective index values satisfy with respect to the threshold TH1 and the threshold TH2.

Here, the ratio L of the index value A3 is L=57%. Therefore, since the ratio L of the index value A3 corresponds to the condition 2, the processor determines that the calibration data D1 is updated every other day. The ratio L of the index value B3 is L=43%. Therefore, since the ratio L of the index value B3 corresponds to the condition 2, the processor determines that the calibration data D2 is updated every other day. Further, the ratio L of the index value C3 is L=0%. Therefore, since the ratio L of the index value C3 corresponds to the condition 3, the processor determines that the calibration data D3 is updated every three days. After the update frequency is determined, the flow of FIG. 5 ends.

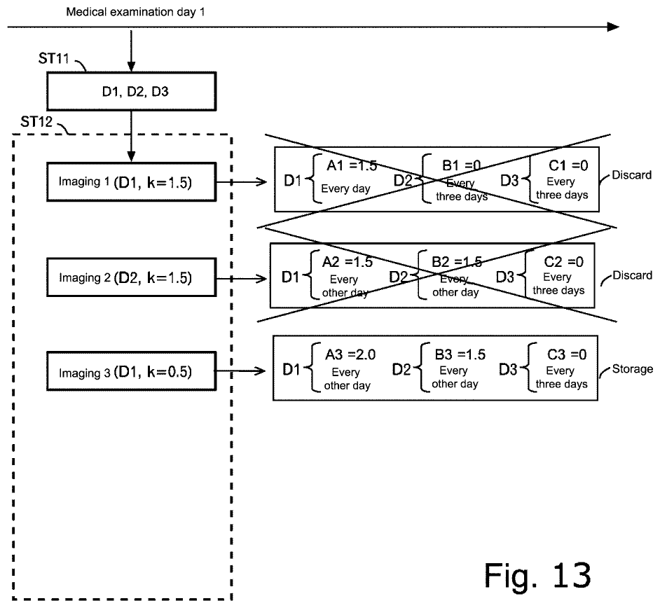

After determining the update frequency, the processor stores the index value and the update frequency obtained by the flow of FIG. 5 in the storage device. FIG. 13 illustrates the index values and update frequencies of the calibration data D1, D2, and D3 stored in association with the imaging 3. Note that the index value and the update frequency obtained in the previous imaging 2 are discarded because they are old information.

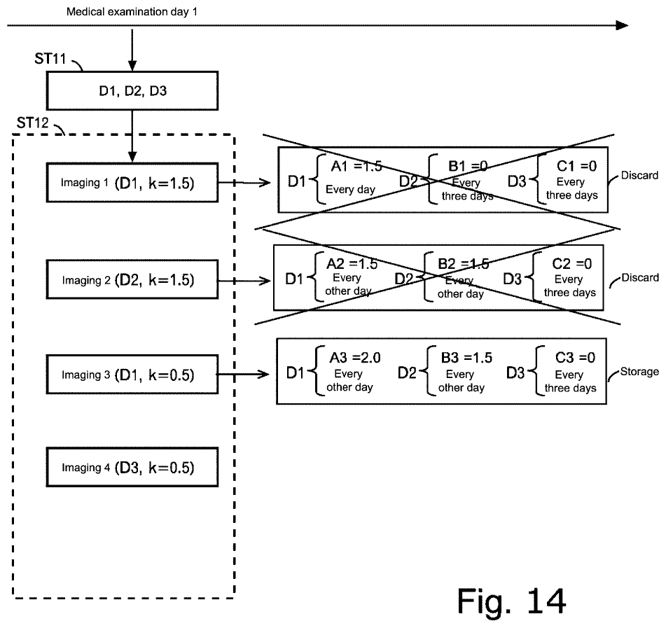

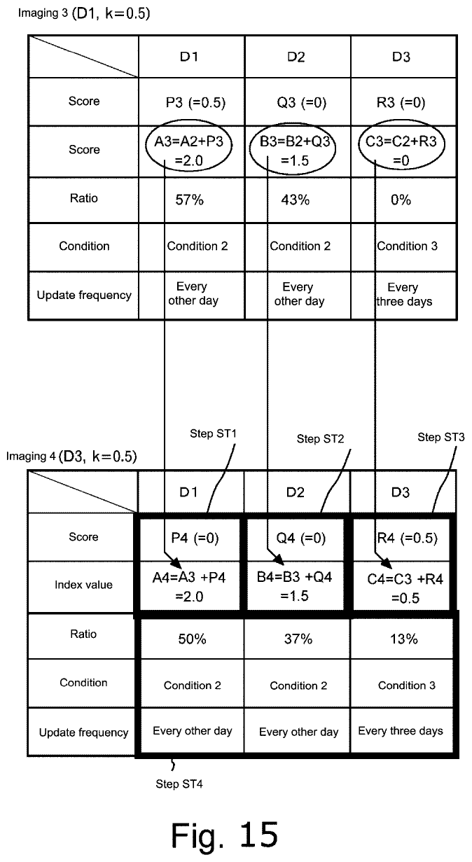

Next, the operator operates the console to input a signal for selecting a protocol corresponding to the imaging purpose of the imaging 4. When this signal is input, the processor selects a protocol corresponding to the imaging purpose of the imaging 4 from among a plurality of protocols. After the protocol is selected, imaging 4 is executed as illustrated in FIG. 14. When the imaging 4 is executed, the processor determines the update frequencies of the calibration data D1, D2, and D3. A method of determining the update frequency will be described below with reference to FIG. 15 together with the flow of FIG. 5.

FIG. 15 is a diagram illustrating a table for describing each step of FIG. 5 when the imaging 4 is executed. In FIG. 14, not only is the table of the imaging 4 shown but also the table of the imaging 3.

In step ST1, an index value serving as a criterion for determining the update frequency of the calibration data D1 is determined. Specifically, the index value is determined as follows. In step ST1, first, a score P4 of the calibration data D1 for the imaging 4 is determined. When the calibration data D1 is not used in the imaging 4, the processor assigns P4=0 to the score P4. On the other hand, when the calibration data D1 is used in the imaging 4, the processor assigns the value of the weighting coefficient k to the score P4. In the imaging 4, since the calibration data D1 is not used, the processor assigns P4=0 to the score P4.

After determining the score P4, the processor determines an index value A4 serving as a criterion for determining the update frequency of the calibration data D1 based on the score P4. Specifically, the processor calculates an index value A4 (=A3+P4) by adding the score P4 to the index value A3 determined in the previous imaging 3. Therefore, A4 is expressed by the following formula.

$$A 4 = A 3 + P 4 = 2. + 0 = 2.$$

Therefore, the index value A4 is calculated as A4=2.0. Once the index value A4 has been calculated, the process proceeds to step ST2.

In step ST2, an index value is determined as a criterion for determining the update frequency of the calibration data D2. Specifically, the index value is determined as follows. In step ST2, first, a score Q4 of the calibration data D2 for the imaging 4 is determined. When the calibration data D2 is not used in the imaging 4, the processor assigns Q4=0 to the score Q4. On the other hand, when the calibration data D2 is used in the imaging 4, the processor assigns the value of the weighting coefficient k to the score Q4. In the imaging 4, since the calibration data D2 is not used, the processor assigns Q4=0 to the score Q4.

When the score Q4 is determined, the processor determines the index value B4 serving as a criterion for determining the update frequency of the calibration data D3 based on the score Q4. Specifically, the processor calculates the index value B4 (=B3+Q4) by adding the score Q4 to the index value B3 determined in the imaging 3. B4 is expressed by the following formula.

$$B 4 = B 3 + Q 4 = 1.5 + 0 = 1.5$$

Therefore, the index value B4 is calculated as B4=1.5. Once the index value B4 has been calculated, the process proceeds to step ST3.

In step ST3, the processor determines an index value serving as a criterion for determining the update frequency of the calibration data D3. Specifically, the index value is determined as follows. In step ST3, the processor determines a score R4. When the calibration data D3 is not used in the imaging 4, the processor assigns R4=0 to the score R4. On the other hand, when the calibration data D3 is used in the imaging 4, the processor assigns the value of the weighting coefficient to the score R4. Since the calibration data D3 is used in the imaging 4, the processor assigns the value of the weighting coefficient k to the score R4. In the imaging 4, the weighting coefficient k for the imaging purpose is k (=0.5). Therefore, the processor assigns R4=0.5 to the score R4.

When the score R4 is determined, the processor determines the index value C4 serving as a criterion for determining the update frequency of the calibration data D3 based on the score R4. Specifically, the processor calculates the index value C4 (=C3+R4) by adding the score R4 to the index value C3 determined in the imaging 3. C4 is a value expressed by the following formula.

$$C 4 = C 3 + R 4 = 0 + 0.5 = 0.5$$

Therefore, the index value C4 is determined as C4=0.5.

Therefore, at the time point when the imaging 4 ends, the index value A4=2.0, the index value B4=1.5, and the index value C4=0.5 are determined as the latest index values. After calculating index values A4, B4, and C4, processing proceeds to step ST4.

In step ST4, the processor determines update frequencies of the calibration data D1, D2, and D3 based on the index values A4, B4, and C4. Specifically, the update frequencies of the calibration data D1, D2, and D3 are determined as follows. First, the processor calculates a ratio L (%) of each index value to the total value of the index values A4, B4, and C4. Here, since the index value A4=2.0, the index value B4=1.5, and the index value C4=0.5, the total value of the index values is 4.0. Therefore, the ratio L of each index value is 50% for the index value A4, 37% for the index value B4, and 13% for the index value C4. Then, the processor compares the ratios of the respective index values with the thresholds, and determines which condition among the conditions 1 to 3 the ratios L of the respective index values satisfy with respect to the threshold TH1 and the threshold TH2.

Here, the ratio L of the index value Ae is L=50%. Therefore, since the ratio L of the index value A4 corresponds to the condition 2, the processor determines that the calibration data D1 is updated every other day. The ratio L of the index value B4 is L=37%. Therefore, since the ratio L of the index value B4 corresponds to the condition 2, the processor determines that the calibration data D2 is updated every other day. Further, the ratio L of the index value C4 is L=3%. Therefore, since the ratio L of the index value C4 corresponds to the condition 3, the processor determines that the calibration data D3 is updated every three days. After the update frequency is determined, the flow of FIG. 5 ends.

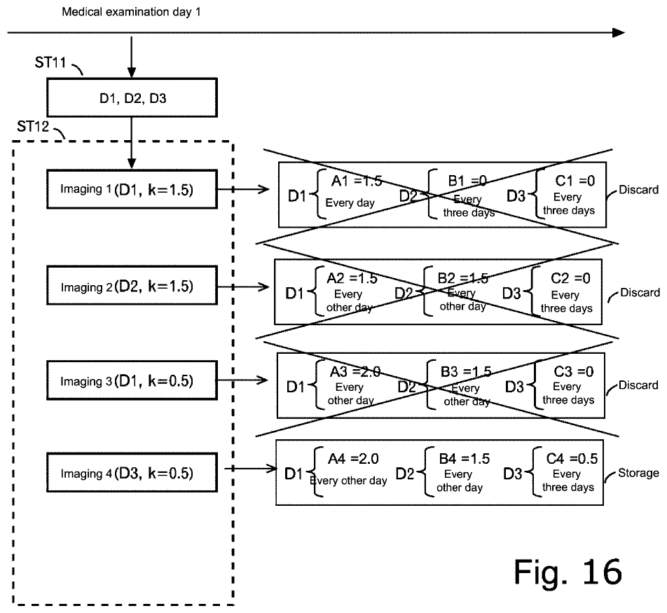

After determining the update frequency, the processor stores the index value and the update frequency obtained by the flow of FIG. 5 in the storage device. FIG. 16 illustrates the index values and update frequencies of the calibration data D1, D2, and D3 stored in association with the imaging 4. Note that the index value and the update frequency obtained in the previous imaging 3 are discarded because they are old information.

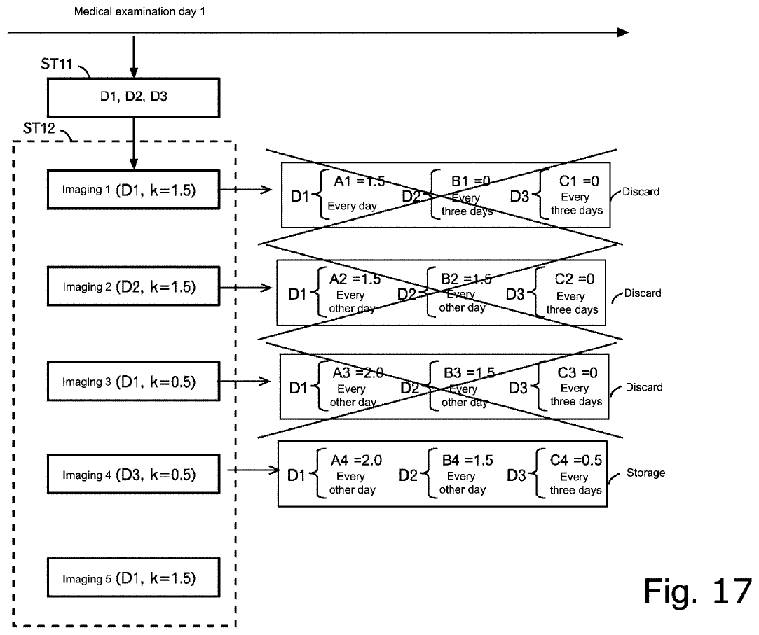

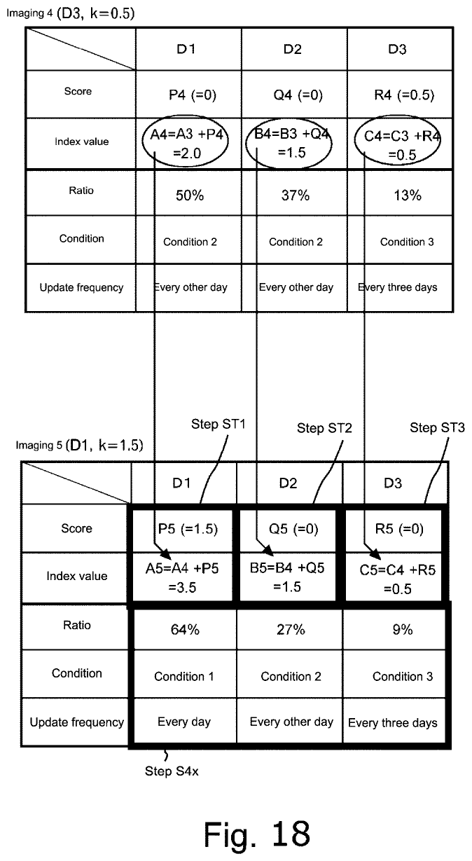

Next, the operator operates the console to input a signal for selecting a protocol corresponding to the purpose of the imaging 5. When this signal is input, the processor selects a protocol corresponding to the imaging purpose of the imaging 5 from among a plurality of protocols. After the protocol is selected, the imaging 5 is executed as illustrated in FIG. 17. When the imaging 5 is executed, the processor determines the update frequencies of the calibration data D1, D2, and D3. A method of determining the update frequency will be described below with reference to FIG. 18 together with the flow of FIG. 5.

FIG. 18 is a diagram illustrating a table for describing each step of FIG. 5 when the imaging 5 is executed. In FIG. 18, not only is the table of the imaging 5 shown but also the table of the imaging 4.

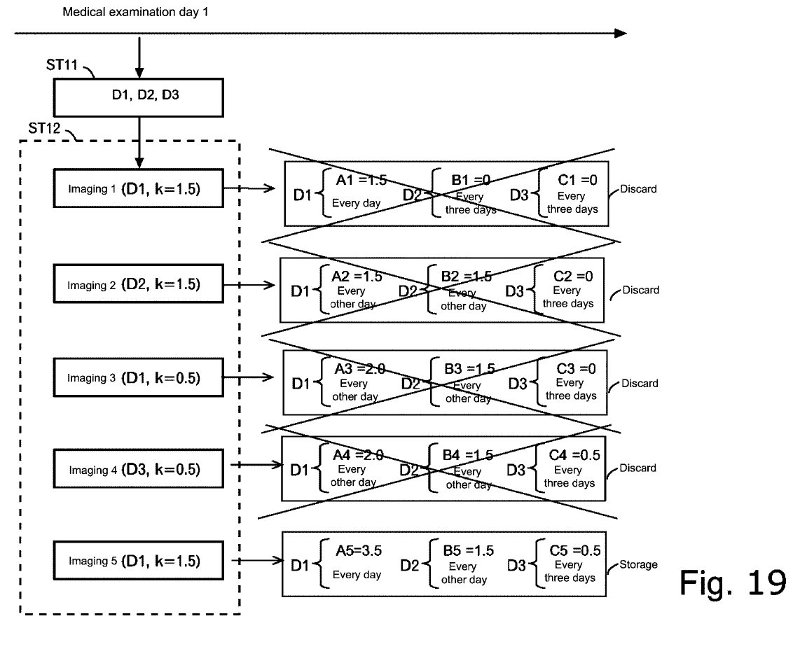

FIG. 18 illustrates the update frequency determined by executing steps ST1 to ST4 for the imaging 5. Since the method of determining the update frequency is as described above, description of FIG. 18 is omitted. In the imaging 5, the calibration data D1 is determined to be updated every day, the calibration data D2 is determined to be updated every other day, and the calibration data D3 is determined to be updated every three days. The processor stores the index values A5, B5, and C5 obtained for the imaging 5 and the update frequencies “daily,” “every other day,” and “every three days” in the storage device. FIG. 19 illustrates the index values and update frequencies of the calibration data D1, D2, and D3 stored in association with the imaging 5. Note that the index value and the update frequency obtained in the previous imaging 4 are discarded because they are old information.

Therefore, on medical examination day 1, the latest index values of calibration data D1, D2, and D3 are A5=3.5, B5=1.5, and Cg=0.5, respectively, and the latest update frequencies are determined to be “every day,” “every other day,” and “every three days,” respectively. Next, medical examination day 2 will be described.

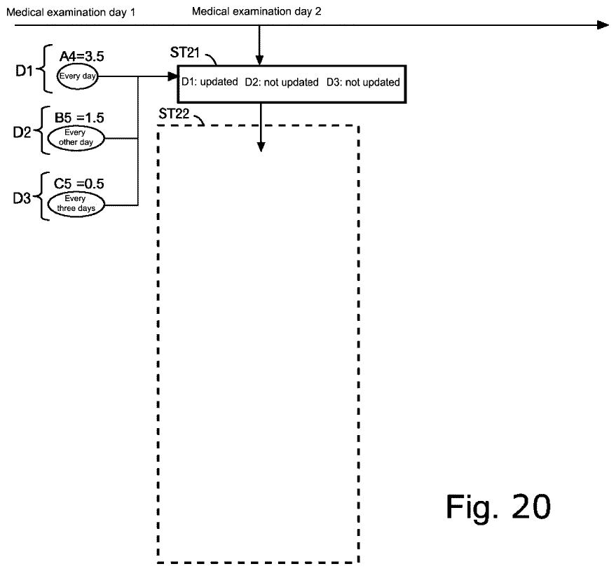

FIG. 20 is an explanatory diagram of medical examination day 2.

Note, for medical examination day 1, the latest index values A5=3.5, B5=1.5, and Cg=0.5 obtained in imaging 5 for calibration data D1, D2, and D3 are shown, along with the latest update frequencies of “every day,” “every other day,” and “every three days.”

On medical examination day 2, in step ST21, the operator inputs a command into the console to execute the calibration. When this instruction is input, the processor reads the latest update frequency stored in the storage device. Here, the latest update frequency of the calibration data D1 is determined to be “every day.” Therefore, the processor determines to update the calibration data D1 on medical examination day 2 as well. Therefore, the processor determines to execute a calibration scan to obtain the calibration data D1.

The most recent update frequency of the calibration data D2 is determined to be “every other day.” Therefore, the processor checks the past update history of the calibration data D2. In the present embodiment, the calibration data D2 is acquired on medical examination day 1, and therefore the calibration data D2 is updated on the day before medical examination day 2 (medical examination day 1). Therefore, the processor determines that the calibration data D2 is not updated on medical examination day 2. Therefore, the processor determines not to execute the calibration scan for acquiring the calibration data D2.

Moreover, the latest update frequency of the calibration data D3 is determined to be “every three days.” Therefore, the processor checks the past update history of the calibration data D3. In the present embodiment, the calibration data D3 is acquired on medical examination day 1, and therefore the calibration data D3 is updated to the day before medical examination day 2 (medical examination day 1). Therefore, the processor determines not to update the calibration data D3 on medical examination day 2. Therefore, the processor determines not to execute a calibration scan to obtain calibration data D3.

As a result, on medical examination day 2, the processor determines to only execute a calibration scan to obtain calibration data D1. Therefore, in step ST21, only the calibration data D1 is acquired.

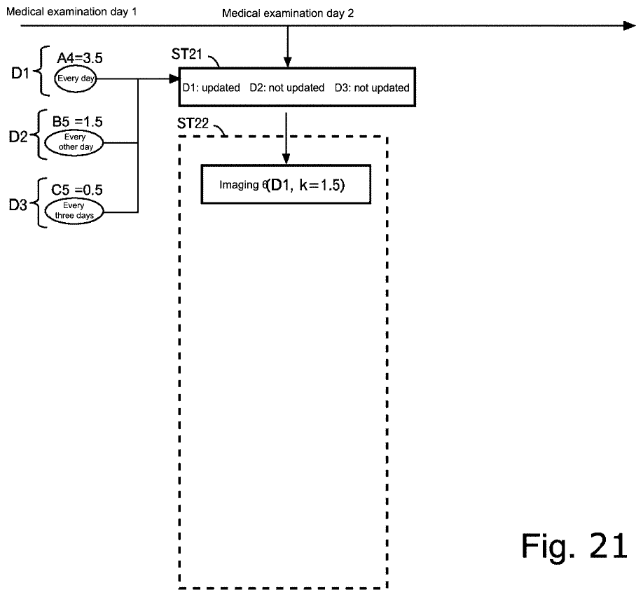

After the calibration data D1 is acquired, imaging of the subject is executed in step ST22. FIG. 21 is an explanatory diagram of step ST22.

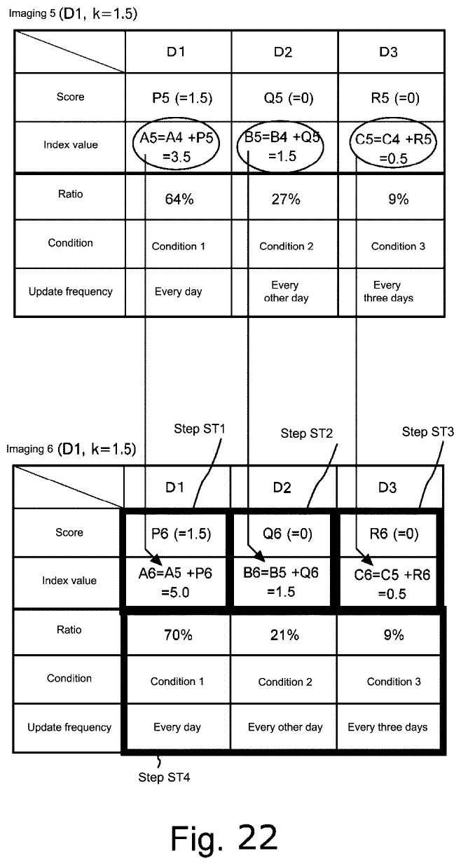

In step ST22, the operator performs an operation on the console to input a signal for selecting the protocol corresponding to the imaging purpose of the imaging 6. When this signal is input, the processor selects a protocol corresponding to the imaging purpose of the imaging 6 from among a plurality of protocols. After the protocol is selected, the imaging 6 is executed. When the imaging 6 is executed, the processor determines update frequencies of the calibration data D1, D2, and D3. A method of determining the update frequency will be described below with reference to FIG. 22 together with the flow of FIG. 5.

FIG. 22 is a diagram illustrating a table for describing each step of FIG. 5 when the imaging 6 is executed. In FIG. 22, not only is the table of the imaging 6 illustrated but also the table of the last imaging 5 on medical examination day 1.

In step ST1, an index value serving as a criterion for determining the update frequency of the calibration data D1 is determined. Specifically, the index value is determined as follows. In step ST1, first, the score P6 of the calibration data D1 for the imaging 6 is determined. When the calibration data D1 is not used in imaging 6, the processor assigns P6=0 to the score P6. On the other hand, when the calibration data D1 is used in the imaging 6, the processor assigns the value of the weighting coefficient k to the score P6. Since the calibration data D1 is used in the imaging 6, the processor assigns the value of the weighting coefficient k to the score P6. In imaging 6, the weighting coefficient k of the imaging purpose is k (=1.5). Therefore, the processor assigns P6=1.5 to score P6.

After determining the score P6, the processor determines an index value A6 that serves as a criterion for determining the update frequency of the calibration data D1, based on the score P6. Specifically, the processor calculates an index value A6 (=A5+P6) by adding the score P6 to the index value A5 determined in the imaging 5. A6 is a value expressed by the following formula.

$$A 6 = A 5 + P 6 = 3.5 + 1.5 = 5.$$

Therefore, the index value A6 is calculated as A6=5.0. After the index value A6 has been calculated, the process proceeds to step ST2.

In step ST2, an index value is determined as a criterion for determining the update frequency of the calibration data D2. Specifically, the index value is determined as follows. In step ST2, first, a score Q6 of the calibration data D2 for the imaging 6 is determined. When the calibration data D2 is not used in the imaging 6, the processor assigns Q6=0 to the score Q6. On the other hand, when the calibration data D2 is used in the imaging 6, the processor assigns the value of the weighting coefficient k to the score Q6. In the imaging 6, since the calibration data D2 is not used, the processor assigns Q6=0 to the score Q6.

When the score Q6 is determined, the processor determines the index value Q6 serving as a criterion for determining the update frequency of the calibration data D3 based on the score B6. Specifically, the processor calculates an index value B6 (=B5+Q6) by adding the score Q6 to the index value B5 determined in the imaging 5. B6 is a value expressed by the following formula.

$$B 6 = B 5 + Q 6 = 1.5 + 0 = 1.5$$

Therefore, the index value B6 is calculated as B6=1.5. After the index value B6 has been calculated, the process proceeds to step ST3.

In step ST3, the processor determines an index value serving as a criterion for determining the update frequency of the calibration data D3. Specifically, the index value is determined as follows. In step ST3, the processor determines a score R6. If the calibration data D3 is not used in imaging 6, the processor assigns R6=0 to the score R6. On the other hand, when the calibration data D3 is used in the imaging 6, the processor assigns a value of a weighting coefficient to the score R6. Since the calibration data D3 is not used in the imaging 6, the processor determines the score R6 as R6=0.

When the score R6 is determined, the processor determines the index value C6 serving as a criterion for determining the update frequency of the calibration data D3 based on the score R6. Specifically, the processor calculates the index value C6 (=C5+R6) by adding the score R6 to the index value C5 determined in the imaging 5. C6 is a value expressed by the following formula.

$$C 6 = C 5 + R 6 = 0.5 + 0 = 0$$

Therefore, in the imaging 6, the index value A6=5.0, the index value B6=1.5, and the index value C6=0.5 are determined. After calculating the index values A6, B6, and C6, the process proceeds to step ST4.

In step ST4, the processor determines update frequencies of the index values A6, B6, and C6 based on the calibration data D1, D2, and D3. Specifically, the update frequencies of the calibration data D1, D2, and D3 are determined as follows. First, the processor calculates a ratio L (%) of each index value to the total value of the indicator values A6, B6, and C6. Here, since the index value A6=5.0, the index value B6=1.5, and the index value C5=0.5, the total value of the index values is 7.0. Therefore, the ratio L of each index value is 70% for the index value A6, 21% for the index value B6, and 9% for the index value C6. Then, the processor compares the ratios of the respective index values with the thresholds, and determines which condition among the conditions 1 to 3 the ratios P of the respective index values satisfy with respect to the threshold TH1 and the threshold TH2.

Here, the ratio L of the index value A6 is L=70%. Therefore, since the ratio L of the index value A6 corresponds to the condition 1, the processor determines that the calibration data D1 is updated every day. The ratio L of the index value B6 is L=21%. Therefore, since the ratio L of the index value B6 corresponds to the condition 2, the processor determines that the calibration data D2 is updated every other day. Further, the ratio L of the index value C6 is L=9%. Therefore, since the ratio L of the index value C6 corresponds to the condition 3, the processor determines that the calibration data D3 is updated every three days. After the update frequency is determined, the flow of FIG. 5 ends.

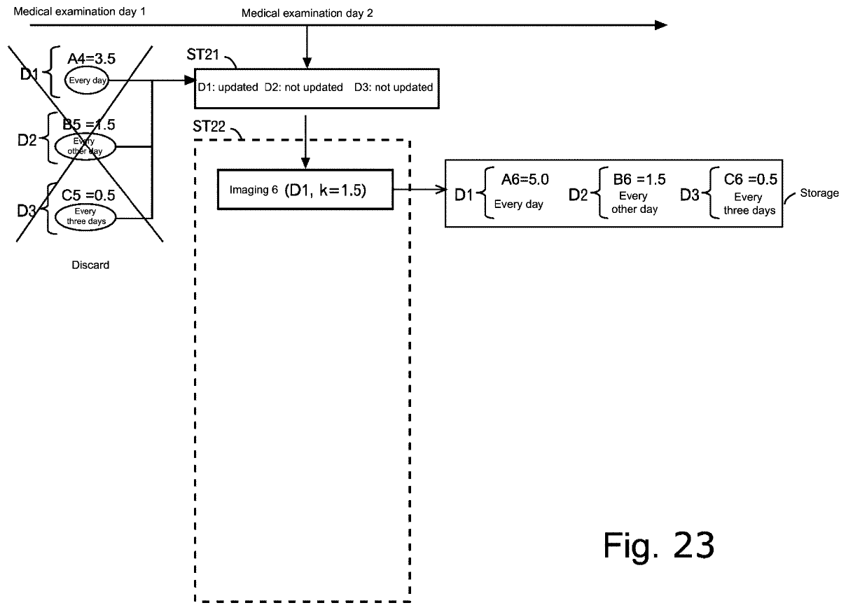

After determining the update frequency, the processor stores the index value and the update frequency obtained by the flow of FIG. 5 in the storage device. FIG. 23 illustrates the index values and update frequencies of the calibration data D1, D2, and D3 stored in association with the imaging 6. Note that the index value and the update frequency obtained in the previous imaging 5 are discarded because they are old information. Similarly, the next imaging is waited for, and the update frequency is determined by executing the flow of FIG. 5 every time the imaging is executed.

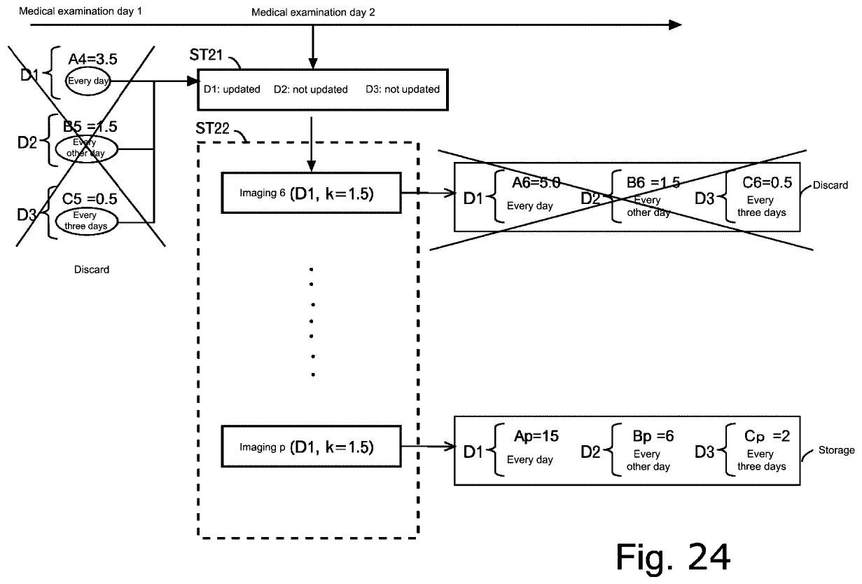

FIG. 24 is a diagram illustrating the index values and update frequencies obtained in a final imaging p on medical examination day 2. On medical examination day 2, the latest index value of the calibration data D1, D2, and D3 are Ap=15, Bp=6, and Cp=2, respectively, and the latest update frequencies are determined to be “every day,” “every other day,” and “every three days,” respectively.

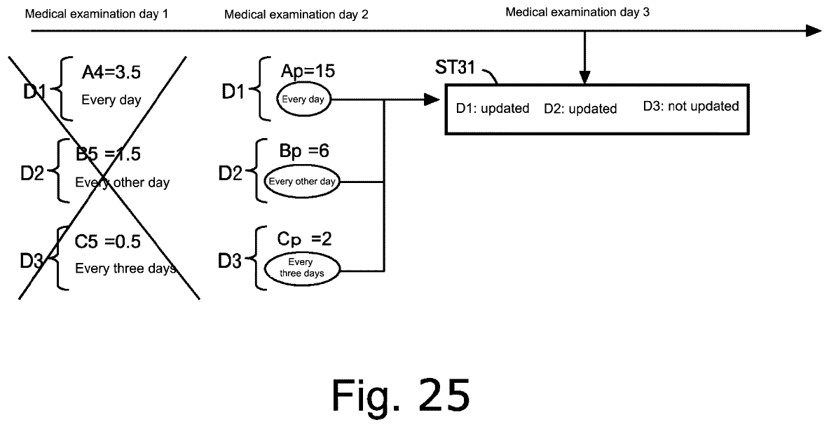

Next, medical examination day 3 will be described. FIG. 25 is an explanatory diagram of medical examination day 3. For medical examination day 2, the latest index values Ap=15, Bp=6, and Cp=2 obtained in the imaging p for the calibration data D1, D2, and D3, and the latest update frequencies “every day,” “every other day,” and “every three days” are shown. On medical examination day 3, in step ST31, the operator inputs a command into the console to execute the calibration. When this instruction is input, the processor reads the latest update frequency stored in the storage device. Here, the latest update frequency of the calibration data D1 is determined to be “every day.” Therefore, the processor determines to update the calibration data D1 on medical examination day 3 as well. Therefore, the processor determines to execute a calibration scan to obtain the calibration data D1.

The most recent update frequency of the calibration data D2 is determined to be “every other day.” Therefore, the processor checks the past update history of the calibration data D2. In the present embodiment, the calibration data D2 is acquired on medical examination day 1, and is not acquired on medical examination day 2. Therefore, the processor determines to update the calibration data D2 on medical examination day 3. Therefore, the processor determines to execute a calibration scan to obtain the calibration data D2.

Moreover, the latest update frequency of the calibration data D3 is determined to be “every three days.” Therefore, the processor checks the past update history of the calibration data D3. In the present embodiment, the calibration data D3 is acquired on medical examination day 1, and is not acquired on medical examination day 2. Therefore, the processor determines not to update the calibration data D3 on medical examination day 3. Therefore, the processor determines not to execute a calibration scan to obtain calibration data D3.

As a result, on medical examination day 3, the processor determines to execute only a calibration scan to obtain calibration data D1 and a calibration scan to obtain calibration data D2. Therefore, in step ST31, only the calibration data D1 and D2 are acquired. After acquiring the calibration data D1 and D2, imaging of the subject is executed in the same manner thereafter, and the update frequency of the calibration data is determined every time imaging of the subject is executed.

In the present embodiment, when determining the update frequency, the processor assigns a weighting coefficient k to the score for calibration data that was used at the time of imaging, while assigning a score of zero to calibration data that was not used at the time of imaging. Then, the score and the index value obtained in the previous imaging are added to calculate the latest index value, and the update frequency is determined for each calibration data based on the latest index value. Therefore, every time the imaging is executed, an index value that reflects the number of times the calibration data has been used can be calculated. Further, in the present embodiment, the weighting coefficient k assigned to the score varies depending on the purpose of imaging. Therefore, an index value reflecting the weighting coefficient k can be calculated every time imaging is executed. That is, in the present embodiment, the update frequency of the calibration data is determined not by focusing only on the number of times the calibration data is used, but by taking into consideration both the number of times the calibration data is used and the weighting coefficient k. Therefore, when the number of times the calibration data is used and the weighting coefficient k of the imaging in which the calibration data is used are low, the frequency of updating the calibration data decreases, thereby shortening the time required for one calibration of the CT system.

Further, in the present embodiment, as described above, the purpose of imaging of the calibration data can be reflected in the update frequency. For example, in imaging requiring high image quality, such as imaging to obtain images of three time phases of the liver, the weighting coefficient k can be set to a high value, and in imaging requiring not so high image quality, such as imaging to obtain images of blood vessels, the weighting coefficient k can be set to a low value. Therefore, since the update frequency of the calibration data can be increased in imaging requiring high image quality, and the update frequency of the calibration data can be decreased in imaging requiring not so high image quality, the update frequency can be determined in consideration of the balance between the time required for calibration and the required image quality.

In addition, even when imaging requiring high image quality is not executed frequently, the weighting coefficient k can be set to a high value to increase the frequency of updating the calibration data, thereby maintaining high image quality.

Furthermore, in imaging that does not require a very high image quality, such as imaging to obtain a blood vessel image, the weighting coefficient k is set to a low value. However, in the present embodiment, as described above, when determining the update frequency of the calibration data, the number of times the calibration data is used is also taken into consideration. Therefore, even when the image quality required for imaging is not so high, when the demand for imaging is high, the frequency of updating the calibration data can be increased.

Note that in the present embodiment, when the calibration data is not used in imaging, zero is assigned to the score, but a value other than zero may be assigned. Furthermore, when calibration data is used in imaging, the score is assigned the value of the weighting coefficient k, but as long as it reflects the value of the weighting coefficient k, the score need not necessarily be assigned the value of the weighting coefficient k; for example, a multiple of the weighting coefficient k may be used as the score value.

In the present embodiment, the update frequency of the calibration data is “every day,” “every other day,” or “every three days,” but the update frequency is not limited to “every day,” “every other day,” or “every three days.” For example, “half a day” or “every four days” may be added to the update frequency.

Note that in the present embodiment, an example is described in which the CT system 10 is used as the medical system. However, the present invention is not limited to the CT system 10 and can be applied to a system other than the CT system 10 (for example, PET-CT system), so long as the medical system irradiates an X-ray source onto the subject 112.

1. A medical system for performing an imaging to obtain medical images of a subject, the medical system including one or more processors, wherein the one or more processors, each time an imaging is executed, execute the following operations:
selecting calibration data to be used to reconstruct an image from a plurality of calibration data; and
determining an update frequency for each calibration data based on the number of times each calibration data is used and a weighting coefficient according to the imaging purpose of the executed imaging.

2. The medical system according to claim 1, wherein the one or more processors determine an update frequency for each calibration data each time imaging is executed.

3. The medical system according to claim 2, wherein the one or more processors calculate, for each calibration data item, an index value reflecting a number of times the calibration data item has been used and a weighting coefficient according to a purpose of imaging the subject, each time imaging is executed.

4. The medical system according to claim 3, wherein the one or more processors determine a score for each calibration data each time imaging is executed and calculate the index value based on the score.

5. The medical system according to claim 4, wherein the one or more processors further execute:
assigning a first value to the score according to a weighting coefficient when the calibration data has not been used to reconstruct an image; and
assigning a second value to the score when the calibration data has not been used to reconstruct an image.

6. The medical system according to claim 5, wherein the first value is a weighting coefficient value and the second value is zero.

7. The medical system according to claim 4, wherein, when one imaging is executed, the one or more processors execute:
determining a first score for each piece of calibration data;
calculating, for each piece of calibration data, a first index value reflecting the number of times the calibration data has been used and a weighting coefficient based on the first score; and
determining a first update frequency for each piece of calibration data based on the first index value, and
when a next imaging is executed after the one imaging, the one or more processors execute:
determining a second score for each piece of calibration data;
calculating, for each piece of calibration data, a second index value reflecting the number of times the calibration data has been used and a weighting coefficient based on the second score and the first index value; and
determining a second update frequency for each piece of calibration data based on the second index value.

8. The medical system according to claim 7, wherein, when the second index value and the second update frequency are determined, the one or more processors store the second index value and the second update frequency and discard the first index value and the first update frequency.

9. The medical system according to claim 7, wherein the one or more processors calculate the second index value by adding the second score and the first index value.

10. The medical system according to claim 1, wherein:
a storage device of the medical system or a storage device accessible to the medical system stores a plurality of calibration data obtained by a plurality of calibration scans; and
the one or more processors determine an update frequency for each of the plurality of calibration data.

11. The medical system according to claim 1, wherein:
the one or more processors select a protocol corresponding to an imaging purpose from among a plurality of protocols based on a signal input from a console; and
a weighting coefficient corresponding to the imaging purpose is set for each protocol.

12. The medical system according to claim 1, wherein calibration data is acquired by executing a calibration scan in which a tube voltage applied to an X-ray tube is switched between a first tube voltage and a second tube voltage.

13. The medical system according to claim 1, wherein:
the latest update frequency of each calibration data is stored in a memory device of the medical system or a memory device accessible to the medical system; and
when a command to execute calibration is input from a console, the one or more processors determine whether to update each calibration data based on the latest update frequency stored in the memory device.
